(19) United States
(12) Patent Application Publication
(10) Pub. No.: US 2011/0009480 A1
(43) Pub. Date: Jan. 13, 2011

(54) Biologically active taxane analogs and methods of treatment

(75) Inventors: James D. McChesney, Boulder, CO (US); Rodger Lamb, Westminster, CO (US); Stanley Kahler, Litteton, CO (US)

Correspondence Address: MORGAN, LEWIS & BOCKIUS LLP, 1701 MARKET STREET, PHILADELPHIA, PA, 19103-2921, US

(73) Assignee: Tapestry Pharmaceuticals, Inc., Boulder, CO (US)

(21) Appl. No.: 12/803,625

(22) Filed: Apr. 9, 2010

Related U.S. Application Data
(63) Continuation of application No. 11/834,489, filed on Aug. 6, 2007, now abandoned.
Application No. 11/834,489 is a continuation of application No. 11/680,563, filed on Feb. 28, 2007, now abandoned.

(51) Int. Cl. A61K 31/357; C07D 493/04; A61P 35/00

(52) U.S. Cl. 514/452; 549/358

(57) ABSTRACT

The present application relates to new taxane analogs, pharmaceutical compositions comprising such analogs and methods of treating cancer comprising such compositions. The compounds according to the present application have the general formula:

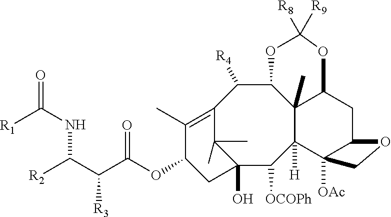

wherein R₁ and R₂ are each selected from H, alkyl, alkenyl or aryl; R₃ is hydroxyl or OP₁; R₄ is OH or R₇COO; R₇ is alkyl, alkenyl or aryl, R₈ and R₉ are each independently selected from H, alkyl or alkenyl. The compounds of the present application may particularly be 9,10-α,α-OH taxane analogs that are formed by a process starting with a standard taxane as the starting compound.

### CROSS REFERENCE TO RELATED APPLICATION

This application is a continuation of U.S. Ser. No. 11/680,563, filed Feb. 28, 2007.

### FIELD OF THE INVENTION

The present application generally relates to chemical compounds for use in treating cancer patients. More particularly, the present application is directed to new and useful taxane analogs and further to methods for producing them. The present application is also directed to pharmaceutical formulations comprising the disclosed taxanes and methods of treating cancer with the disclosed taxanes and their pharmaceutical formulations. Specifically, the present application relates to 9,10-α,α-OH taxane analogs, production methods and intermediates useful in the formation thereof.

### BACKGROUND OF THE INVENTION

Various taxane compounds are known to exhibit anti-tumor activity. As a result of this activity, taxanes have received increasing attention in the scientific and medical community, and are considered to he an exceptionally promising family of cancer chemotherapeutic agents. For example, various taxanes such as paclitaxel and docetaxel have exhibited promising activity against several different varieties of tumors, and further investigations indicate that such taxanes promise a broad range of potent anti-leukemic and tumor-inhibiting activity.

One approach in developing new anti-cancer drugs is the identification of superior analogs and derivatives of biologically active compounds. Modifications of various portions of a complex molecule may lead to new and better drugs having improved properties such as increased biological activity, effectiveness against cancer cells that have developed multi-drug resistance (MDR), fewer or less serious side effects, improved solubility characteristics, better therapeutic profile and the like.

In view of the promising anti-tumor activity of the taxane family, it is desirable to investigate new and improved taxane analogs and derivatives for use in cancer treatment. One particularly important area is the development of drugs having improved MDR reversal properties. Accordingly, there is a need to provide new taxane compounds having improved biological activity for use in treating cancer. There is also a need to provide methods for forming such compounds. Finally, there is a need for methods of treating patients with such compounds in cancer treatment regimens. The present application is directed to meeting these needs.

### Definitions

As used herein, the term “alkyl”, alone or in combination, refers to an optionally substituted straight-chain or branched-chain alkyl radical having from 1 to 10 carbon atoms (e.g. C₁₋₁₀ alkyl or C₁-C₁₀ alkyl). Examples of alkyl radicals include methyl, ethyl, n-propyl, isopropyl, n-butyl, isobutyl, sec-butyl, tert-butyl, tert-amyl, pentyl, hexyl, heptyl, octyl and the like.

The term “alkenyl”, alone or in combination, refers to an optionally substituted straight-chain or branched-chain hydrocarbon radical having one or more carbon-carbon double-bonds and having from 2 to about 18 carbon atoms, Examples of alkenyl radicals include ethenyl, propenyl, 1,4-butadienyl and the like.

The term “aryl”, alone or in combination, refers to an optionally substituted aromatic ring. The term aryl includes monocyclic aromatic rings, polyaromatic rings and polycyclic ring systems. The polyaromatic and polycyclic rings systems may contain from two to four, more preferably two to three, and most preferably two rings. Examples of aryl groups include six-membered aromatic ring systems, including, without limitation, phenyl, biphenyl, naphthyl and anthryl ring systems. The aryl groups of the present application generally contain from five to six carbon atoms.

The term “alkoxy” refers to an alkyl ether radical wherein the term alkyl is defined as above. Examples of alkoxy radicals include methoxy, ethoxy, n-propoxy, isopropoxy, n-butoxy, iso-butoxy, sec-butoxy, tert-butoxy and the like.

The term “diastereoisomer” refers to any group of four or more isomers occurring in compounds containing two or more asymmetric carbon atoms. Compounds that are stereoisomers of one another, but are not enantiomers are called diastereosiomers.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W,; Wuts, P. G. M. Protective Groups in Organic Synthesis, 4^{th} ed,; Wiley: New York, 2007). Exemplary silyl groups for protection of hydroxyl groups include TBDMS (tert-butyldimethylsilyl), NDMS (2-norbornyldimethylsilyl), TMS (trimethylsilyl) and TES (triethylsilyl). Exemplary NH-protecting groups include benzyloxycarbonyl, t-butoxycarbonyl and triphenylmethyl.

Additional, representative hydroxyl protecting groups also include acetyl, butyl, benzoyl, benzyl, benzyloxymethyl, tetrahydropyranyl, 1-ethoxyethyl, allyl, formyl and the like.

The terms “taxanes,” “taxane agents”, “taxane derivatives,” and “taxane analogs” etc . . . are used interchangeably to mean compounds relating to a class of antitumor agents derived directly or semi-synthetically from Taxus brevifolia, the Pacific yew. Examples of such taxanes include paclitaxel and docetaxel and their natural as well as their synthetic or semi-synthetic derivatives.

The term “baccatin” or “baccatin derivatives” means the taxane derivatives in which the side chain at the 13-position of the taxane skeleton is a hydroxy group and these derivatives are often referred to in the literature as a baccatin or “baccatin I-VII” or the like depending, on the nature of the substituents on the tricyclic rings of the taxane skeleton.

The groups or functional groups described in the present application, including for example, C₁₋₁₀ alkyl, alkoxy, alkenyl, aryl and the like, may be unsubstituted or may be further substituted by one or two substituents. The specific substituents may include, for example, amino, halo (bromo, chloro, fluoro and iodo), oxo, hydroxyl, nitro, C₁₋₁₀ alkyl, C₁₋₁₀ alkoxy, C₁₋₁₀ alkylC(═O)— and the like.

“Pharmaceutically acceptable excipient” or “pharmaceutically acceptable salts” as used herein, means the excipient or salts of the compounds disclosed herein, that are pharmaceutically acceptable and provides the desired pharmacological activity. These excipients and salts include acid addition salts formed with inorganic acids such as hydrochloric acid, hydrobromic acid, phosphoric acid, and the like. The salt may also be formed with organic acids such as acetic acid, propionic acid, hexanoic acid, glycolic acid, lactic acid, succinic acid, malic acid, citric acid, benzoic acid and the like.

### Embodiments and Aspects of the Application

In one particular embodiment of the present application, there is provided a compound as a single diastereoisomer of the formula:

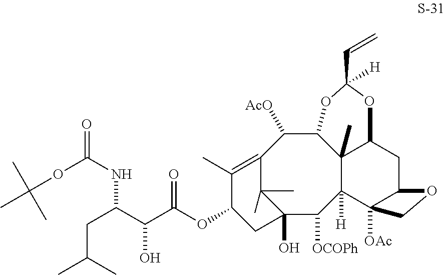

In one particular aspect, the compound is isolated as a pure diastereoisomer. In one variation, the isolated compound is greater than 95% pure. In another variation, the isolated compound is greater than 99% pure.

In another aspect of the present application, there is provided a pharmaceutical composition comprising: a) a therapeutically effective amount of a compound S-31 mentioned above, in the form of a single diasteroisomer; and b) a pharmaceutically acceptable excipient. In another aspect, there is provided a method for the treatment of cancer in a patient comprising administering to the patient a therapeutically effective amount of a compound of the formula:

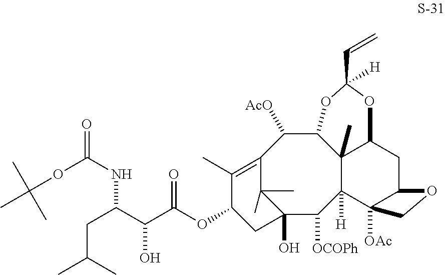

to a patient in need of such treatment. In one variation of the method, the cancer is selected from the group consisting of leukemia, neuroblastoma, glioblastoma, cervical, colorectal, pancreatic, renal, lung, breast, ovarian, prostate, head and neck and melanoma. In another variation of the method, the cancer is colorectal cancer. In a particular variation of the method, the cancer is pancreatic cancer. In another variation of the method, the cancer is neuroblastoma or a glioblastoma.

### SUMMARY OF THE INVENTION

According to the present application, there is provided new and useful compounds for use in cancer treatment having the formula:

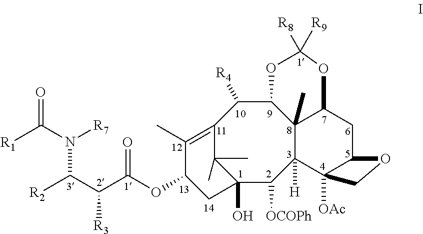

When reference is made to compounds throughout this disclosure, possible R_{X} groups and P_{X} groups are set forth in the following Table 1:

TABLE 1
Rₓ Groups and Pₓ Groups

| | |
|---|---|
| R₁ | C₁-C₆ alkyl, aryl or C₁-C₆ alkoxy |
| R₂ | H, C₁-C₆ alkyl or aryl |
| R₃ | hydroxyl or OP₁, OP₅ or OP₆ |
| R₄ | hydroxyl or R₇COO |
| R₇ | C₁-C₆ alkyl, C₂-C₈ alkenyl or aryl |
| R₈ | H, C₁-C₆ alkyl or C₂-C₈ alkenyl, aryl |
| R₉ | H, C₁-C₆ alkyl or C₂-C₈ alkenyl, aryl |
| P₁ | H or hydroxyl protecting group |
| P₂ | H, hydroxyl protecting group |
| P₃ | H or hydroxyl protecting group including a protecting |
| | group that forms an acetal with P₄ |
| P₄ | H or hydroxyl protecting group including a protecting |
| | group that forms an acetal with P₃ |
| P₅ | H or hydroxyl protecting group |
| P₆ | H or hydroxyl protecting group forming an alkyl, aryl or |
| | substituted aryl acetal with P₇ |
| P₇ | H or nitrogen protecting group forming an alkyl, aryl or |
| | substituted aryl acetal when R₃ is OP₆ |

In one embodiment, R₁ is phenyl or tert-butoxyl, R₂ is phenyl or isobutyl, P₁ and P₂ may each independently be a silyl protecting group such as TBDMS or TES. Compounds according to the present application may be monoacylated at C-10 hydroxy group, such as when R₄ is R₇COO.

Compounds according to the present application have the formula;

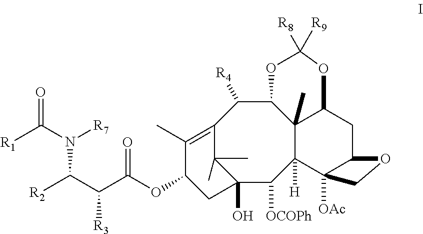

wherein R₁ through R₄ are as defined in Table 1 above and R₈ and R₉ are each independently H, alkyl, alkenyl or aryl. Compounds according to the present application may be monoacylated at C10, such as when R₄ is R₇COO.

For example, in the present application, there is provided compounds of formula:

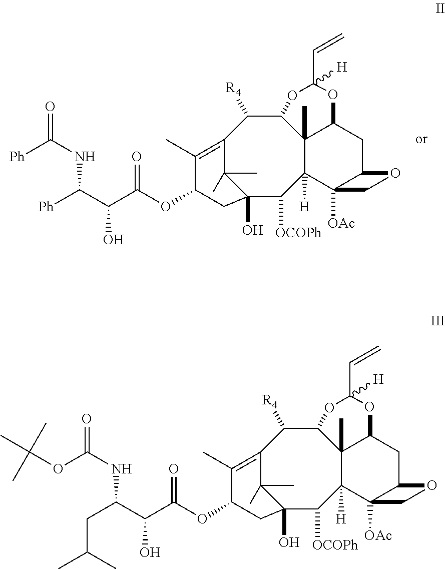

wherein R₄ is hydroxyl or CH₃COO.

Another example of the 7,9-acetal linked compounds of the application have the formula.

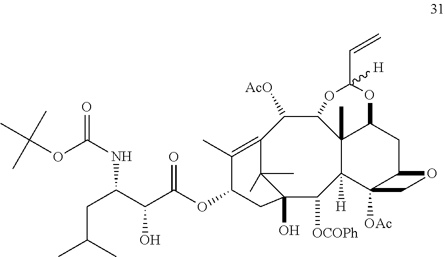

Such compounds include diastereoisomers of the formulae:

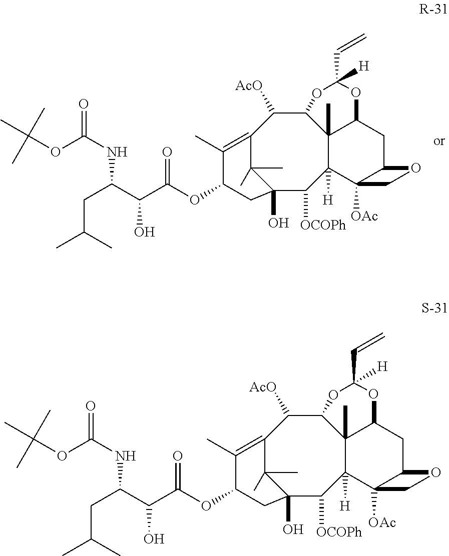

In certain aspects of the above compounds, the compound of each of the above diastereoisomers is isolated and purified to greater than 90% pure, greater than 95% pure, greater than 97% pure or greater than 99.5% pure.

Another example of the 7,9-acetal linked compounds of the application have the formula:

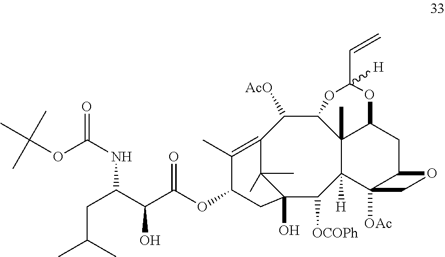

Such compounds include isomers of the formulae:

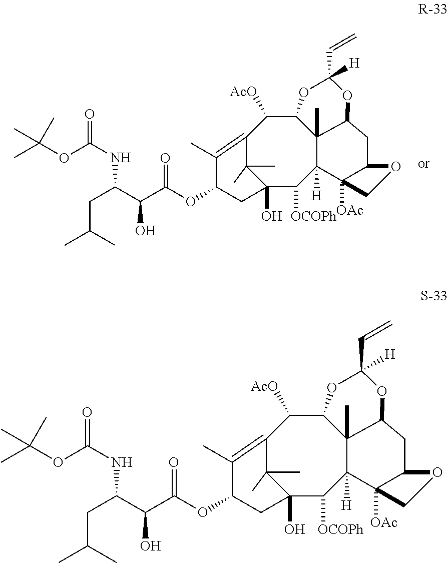

The present application also provides pharmaceutical compositions comprising an isomer of the formula:

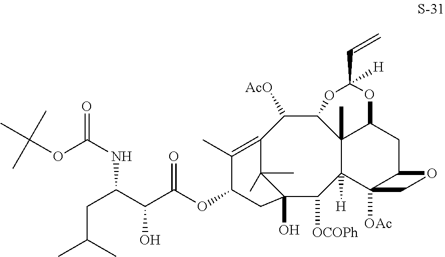

in which the isomer is greater than 90% pure, greater than 95% pure, greater than 97% pure, greater than 99% pure or greater than 99.5% pure.

The present application also provides pharmaceutical compositions comprising a diaster of the formula:

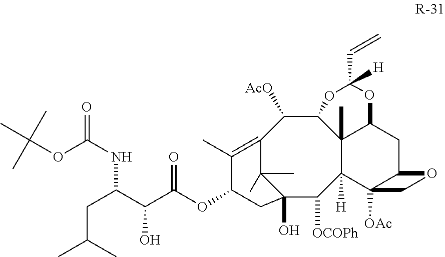

in which the diastereomer is greater than 90% pure, greater than 95% pure, greater than 97% pure or greater than 99% pure. In certain aspects of the above compounds, the purity is determined by HPLC or by isolation of the compound using novel methods described herein.

In addition, the present application provides a method of treating cancer in a patient, comprising administering to the patient a pharmaceutical formulation including a selected concentration of a taxane derivative and a pharmaceutically acceptable carrier therefor, wherein the taxane derivative has a formula:

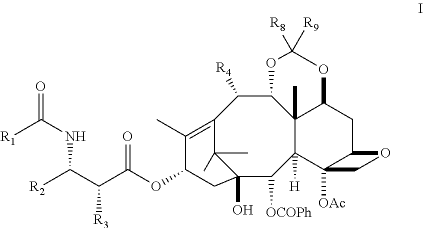

and C-2′ S isomers thereof wherein R₁ through R₉ are as defined in Table 1 above. In one embodiment, the present application provides a method for the treatment of cancer in a patient comprising administering to the patient a composition comprising a compound of formula:

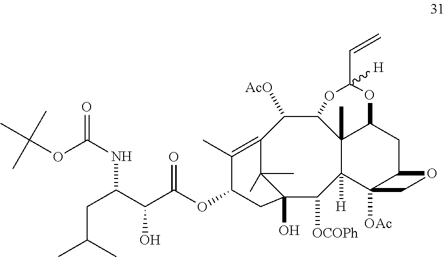

One embodiment includes a method of treating cancer in a patient comprising administering to the patient a composition comprising a compound of formula:

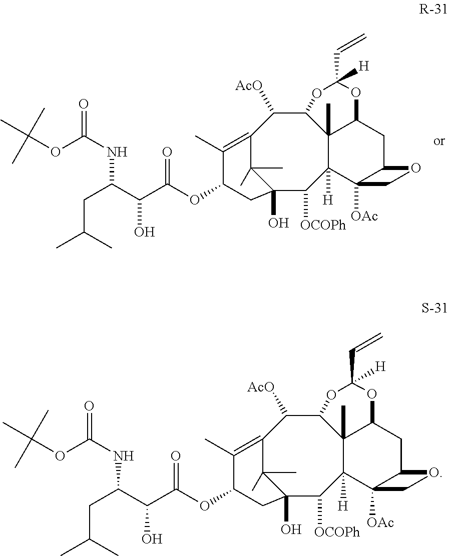

In another embodiment, the present application provides a method for the treatment of cancer in a patient comprising administering to the patient a composition comprising a compound of formula:

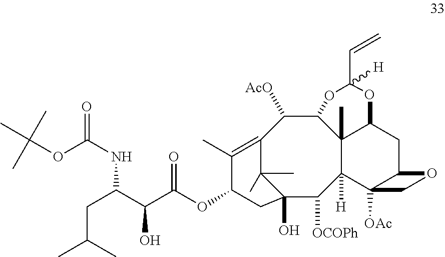

In another embodiment there is also provided a method of treating cancer in a patient comprising administering to the patient a composition comprising a compound of formula:

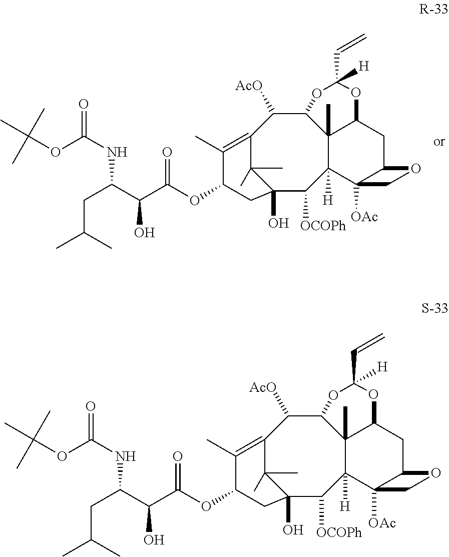

These and other aspects of the present application will become more readily appreciated and understood from a consideration of the following detailed description of the exemplary embodiments of the present application when taken together with the accompanying drawings:

### BRIEF DESCRIPTION OF THE DRAWINGS

FIG. 1 is a representative generalized scheme for forming 9,10-α,α-taxane analogs of the present application.

FIG. 2 is a representative scheme of an exemplary process for the formation of a 7,9-acetal linked compound.

FIG. 3 is a representative scheme of an exemplary process for the deprotection of silyl protected taxanes.

FIG. 4 is a representative scheme of an exemplary process for the formation of a 7,9-acetal linked compound.

FIG. 5 is a representative scheme of an exemplary process for the formation of a 7,9-acetal linked compound,

### DETAILED DESCRIPTION OF THE INVENTION

Paclitaxel and docetaxel have a formula as follows:

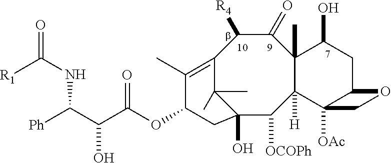

Paclitaxel: R₁=Ph, R₄=AcO

Docetaxel: R₁=t-Butoxy, R₄—OH

Of note is the top part of the molecule illustrated above, which may be seen to have a 9-keto structure and 10-β hydroxy or 10-β acetoxy stereochemistry. The present application provides novel taxane analogs having a stereochemistry at the C-9 and C-10 OH positions of the molecule. Table 2 summarizes the activity of the agents 45, 48, 49, which were found to exhibit excellent inhibition of cell growth against MDR expressing cancer cell lines and a cell line selected for taxanes resistance due to mutant tubulin expression.

Generally, these compounds have been fund to exhibit excellent inhibition of cell growth against MDR expressing cancer cell lines, For example, the 9,10-α,α hydroxy taxane agents discussed in Table 2 exhibit favorable inhibition of cell growth in several of the tested cell lines.

TABLE 2
Biological Activity Data of Selected Taxane Agents

| Cancer Type & Cell line | MDR | Tubulin | Agent | Concentration | Inhibition |
|---|---|---|---|---|---|
| Ovarian Carcinoma | − | Mutant | Paclitaxel | 5 ug/mL | 55% |
| 1A9PTX10 | | | | | |
| Ovarian Carcinoma | − | Mutant | 48 | 0.2 ug/mL | 85% |
| 1A9PTX10 | | | | | |
| Ovarian Carcinoma | − | Mutant | 48 | 0.1 ug/mL | 51% |
| 1A9PTX10 | | | | | |
| Ovarian Carcinoma | − | Mutant | 45 | 0.5 ug/mL | 96% |
| 1A9PTX10 | | | | | |
| Ovarian Carcinoma | − | Mutant | 45 | 0.25 ug/mL | 93% |
| 1A9PTX10 | | | | | |
| Breast Cancer MCF-7 | + | Wild Type | Paclitaxel | 40 ug/mL | 55% |
| NCI-AR | | | | | |
| Breast Cancer MCF-7 | + | Wild Type | 48 | 0.5 ug/mL | 80% |
| NCI-AR | | | | | |
| Breast Cancer MCF-7 | + | Wild Type | 48 | 0.25 ug/mL | 47% |
| NCI-AR | | | | | |
| Breast Cancer MCF-7 | + | Wild Type | 48 | 0.125 ug/mL | 37% |
| NCI-AR | | | | | |
| Breast Cancer MCF-7 | + | Wild Type | 48 | 0.061 ug/mL | 22% |
| NCI-AR | | | | | |
| Breast Cancer MCF-7 | + | Wild Type | 48 | 0.031 ug/mL | 13% |
| NCI-AR | | | | | |
| Breast Cancer MCF-7 | + | Wild Type | 45 | 2.0 ug/mL | 94% |
| NCI-AR | | | | | |
| Breast Cancer MCF-7 | + | Wild Type | 45 | 1.0 ug/mL | 65% |
| NCI-AR | | | | | |
| Breast Cancer MCF-7 | + | Wild Type | 45 | 0.5 ug/mL | 45% |
| NCI-AR | | | | | |
| Breast Cancer MCF-7 | + | Wild Type | 49 | 2.0 ug/mL | 85% |
| NCI-AR | | | | | |
| Breast Cancer MCF-7 | + | Wild Type | 49 | 1.0 ug/mL | 51% |
| NCI-AR | | | | | |
| Breast Cancer MCF-7 | + | Wild Type | 49 | 0.5 ug/mL | 41% |
| NCI-AR | | | | | |
| Neuroblastoma | − | Wild Type | Paclitaxel | 0.1 ug/mL | 54% |
| SK-N-AS | | | | | |
| Neuroblastoma | − | Wild Type | 48 | 0.05 ug/mL | 58% |
| SK-N-AS | | | | | |
| Squamous Cell Carcinoma FADU | − | Wild Type | Paclitaxel | 0.05 ug/mL | 47% |
| Squamous Cell Carcinoma FADU | − | Wild Type | 48 | 0.05 ug/mL | 56% |

The composition of the tested agents were identified as mixtures of the following respective structures:

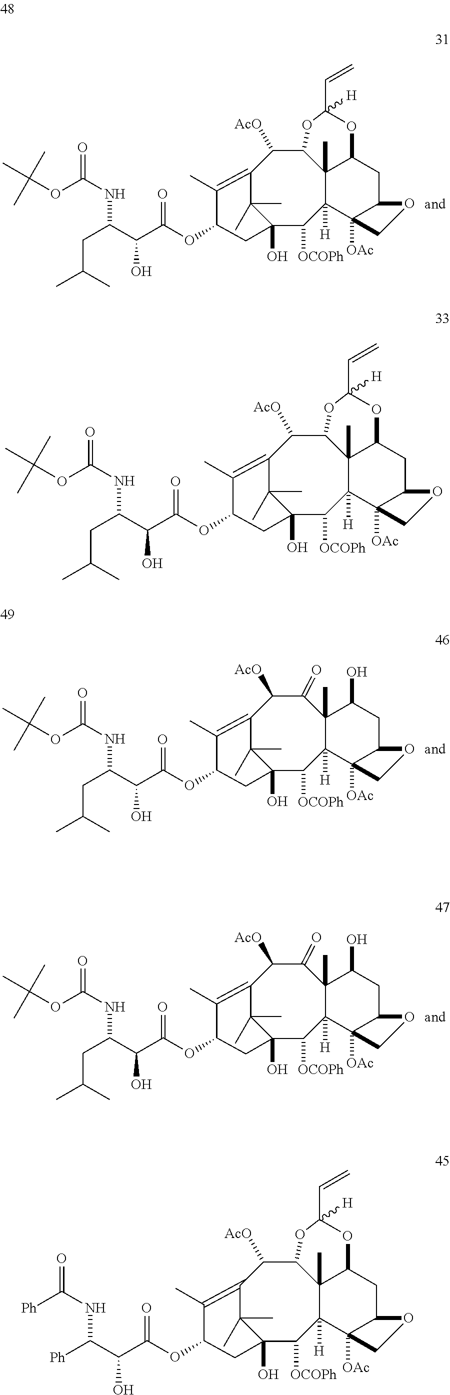

Formula 48 was identified as a mixture of the compounds identified as Formula 31 and Formula 33; consistent with all structures disclosed herein, [symbol] H represents both stereoisomers or diastereoisomers,

Each of the four possible diastereoisomers in the mixture previously identified as Formula 48 in pending U.S. application Ser. No. 10/951,555, filed Sep. 27, 2004, the disclosure of which is incorporated herein in its entirety, were isolated and purified as individual diastereoisomers after intensive investigation. We discovered that standard scale up chromatographic methods for the purification and separation of isomers using silica gel of various grades, from 28-200 mesh, 100-200 mesh, and Davisil® grade 633, 200-425 mesh, C-18 reversed phase media and attempted crystallization with various solvents compositions and solvent mixtures do not provide efficient separation of the isomeric mixture. Solvents such as hexanes, ethyl acetate, methyl tert-butyl ether, ethanol, acetone and their mixtures in different ratios and compositions were determined to be ineffective for the separation of each of the diastereoisomers from each other.

A detailed evaluation of the particular functional groups, including the baccatin hydroxyl group, the baccatin tricyclic ring structure and the side chain in each of the diasteroisomers suggested that a normal phase chromatographic separation of the various stereoisomers might be obtained by the use of a highly efficient: spherical particle silica media and a particular solvent system that would maximize or enhance the interactions between the polar functional groups of the mixture to be separated and the hydrated surface of the silica adsorbent. Among the number of different variables and media that may be employed, a spherical silica and an ether/hydrocarbon elution solvent (MTBE/heptane) was selected for experimentation by TLC. Ultimately, we discovered that a solvent composition of 40% MTBE in heptane gave indication of separation of the diastereoisomers on a silica HPTLC plate with concentration zone with the major compound showing an Rf ˜0.15, Based upon this observation, we elected to attempt a normal phase chromatographic separation of the diastereoisomers by normal phase column chromatography.

Based upon additional biological evaluations, we discovered that the 1″S isomer (i.e. the S diastereoisomer) of the compound of Formula 31 possesses properties that are not obvious in the light of those of the mixture of isomers.

One embodiment of the present application, there is provided the S isomer of Formula 31 (“Formula S-31”), shown below. Also provided herein is a novel method for the preparation of the diastereoisomer, a pharmaceutical composition comprising the isomer and a method of treating cancer comprising administering such a pharmaceutical composition comprising the isomer.

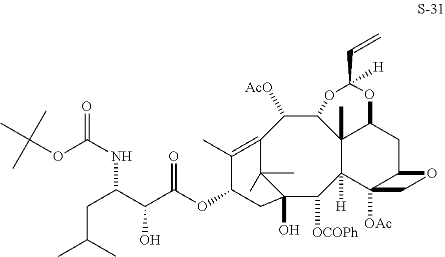

As a result of the large number of stereocenters in the baccatin tricyclic backbone as well as the different stereocenters in the side chain, compounds with the large number of chiral centers such as that of Formula 31 may have a multitude of stereoisomers, and may potentially form a large number of different diastereoisomers. Many of the stereocenters are predisposed in such natural products, while the C9, C10, C2′, C3′ and the acrolein acetal carbon C1 among other asymmetric carbon centers, may form different diastereoisomeric compounds.

In some diastereoisomeric mixtures, one of the two stereoisomers, when isolated using the method disclosed herein is particularly active and an enhancement of the toxicity may be linked to this activity. The other diastereoisomer(s) were found to be markedly less active. For such composition, the gain in activity of one diastereoisomer in a mixture of all other possible diastereoisomers does not compensate for the drawbacks due to a potential for enhanced toxicity of the composition. Analogously, in the mixture of all possible diastereoisomers of Formula 48, one of the diastereoisomers may be primarily responsible for the desirable biological activity of a cancer chemotherapeutic.

From a mixture of the isomers, Formula 48, we discovered that the compounds of Formula 31 show better activity than compounds of Formula 33. Furthermore, in the case of the diastereoisomers of Formula 31, we discovered that the compound of Formula S-31 possesses biological properties that are not obvious in light of those of the mixture of all possible diastereoisomers identified as Formula 48. After having separated and identified all of the individual diastereoisomers, we discovered, surprisingly and unexpectedly, that the S isomer is significantly more active than the isomeric mixture. Further, as shown in Table 3, the compound of Formula S-31 was determined to be significantly more active than the diastereoisomer of Formula R-31 in a number of cytotoxicity assays as measured by MTS proliferation assay.

TABLE 3
MTS Proliferation Assay Results

nM IC₅₀

| Cancer Type & Cell line | S-31 | R-31 |
|---|---|---|
| Neuroblastoma | 11 | 80 |
| SKNAS | | |
| head/neck | 21 | 96 |
| FADU | | |
| prostate | 60 | 212 |
| DU145 | | |
| breast | 10 | 52 |
| MDA435s | | |
| head/neck | 9.4 | 37.7 |
| KB | | |
| head/neck | 553 | 1196 |
| KBV (MDR+) | | |
| colon | 0.006 | 1.6 |
| HT29 | | |
| uterine | 2.9 | 7.8 |
| MESSA | | |
| uterine | 44.8 | 233 |
| MESSA/Dox (MDR+) | | |
| prostate | 0.03 | 2.5 |
| PC-3 | | |

Synthesis of 9,10-α,α-hydroxy taxanes

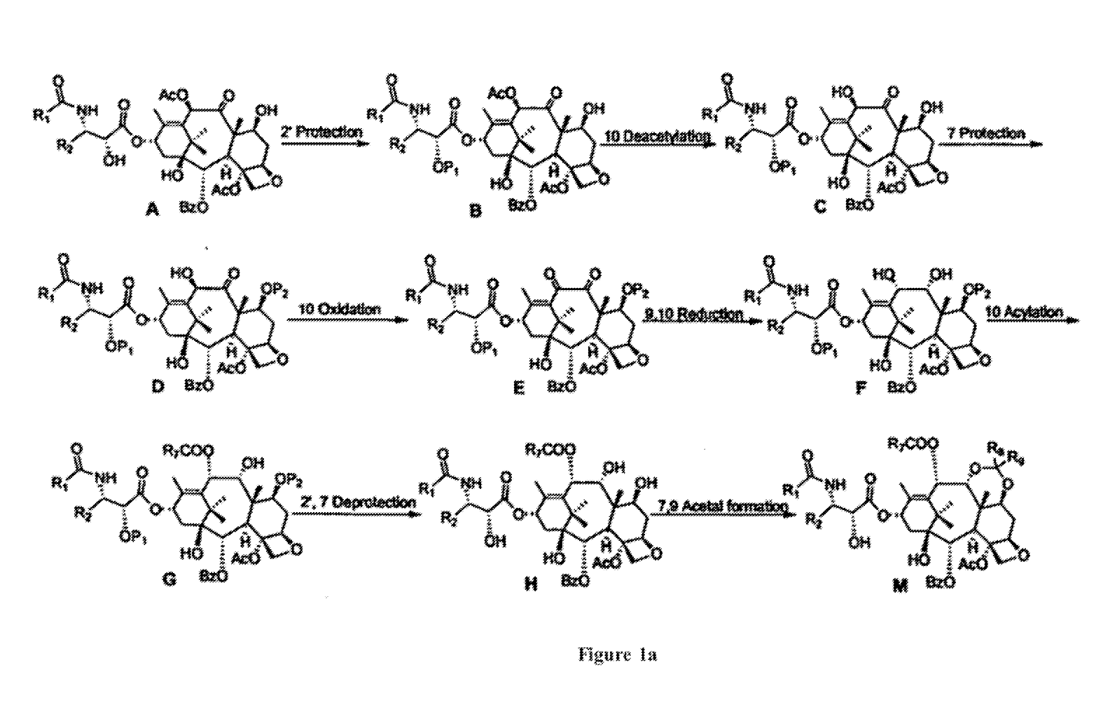

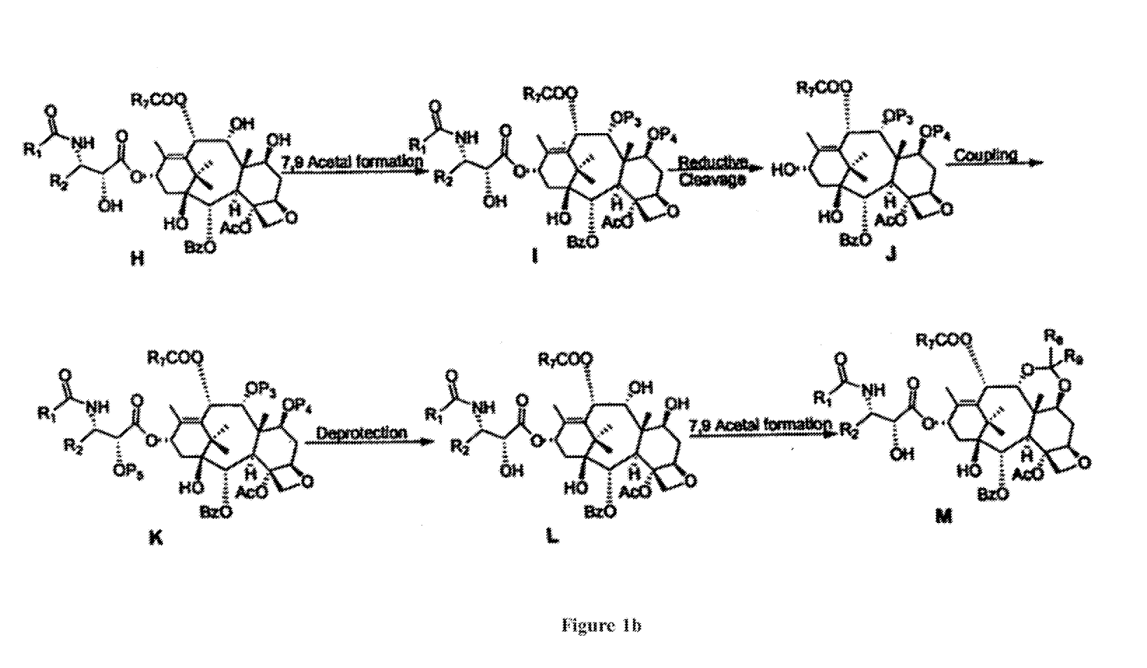

9,10-α,α-hydroxy taxanes may be formed in a number of routes, some of which are disclosed in U.S. Application No, 2005/148657 (U.S. Ser. No. 10/951,555), the complete disclosure of which is incorporated by reference in its entirety. Additionally, as shown in FIG. 1, a 9,10-α,α hydroxy taxane F may be formed directly from a standard taxane A through various transformations, including oxidation of a 10-hydroxy taxane D to a 9,10-diketo taxane E and selective reduction to the 9,10-α,α-hydroxy taxane F. In the compounds shown in FIG. 1, R₁ and R₂ may each be independently H, alkyl such as an isobutyl group or a tert-butyl group, alkenyl such as a tiglyl group, aryl such as a phenyl group, or alkoxy; R₇ may be an alkyl such as a methyl group, alkenyl or aryl; and P₁, P₂, P₃, P₄, and P₅ may each be independently a hydroxyl protecting group, such as a silyl protecting group, including TBDMS or TES, or other hydroxyl protecting groups such as acetals or ethers.

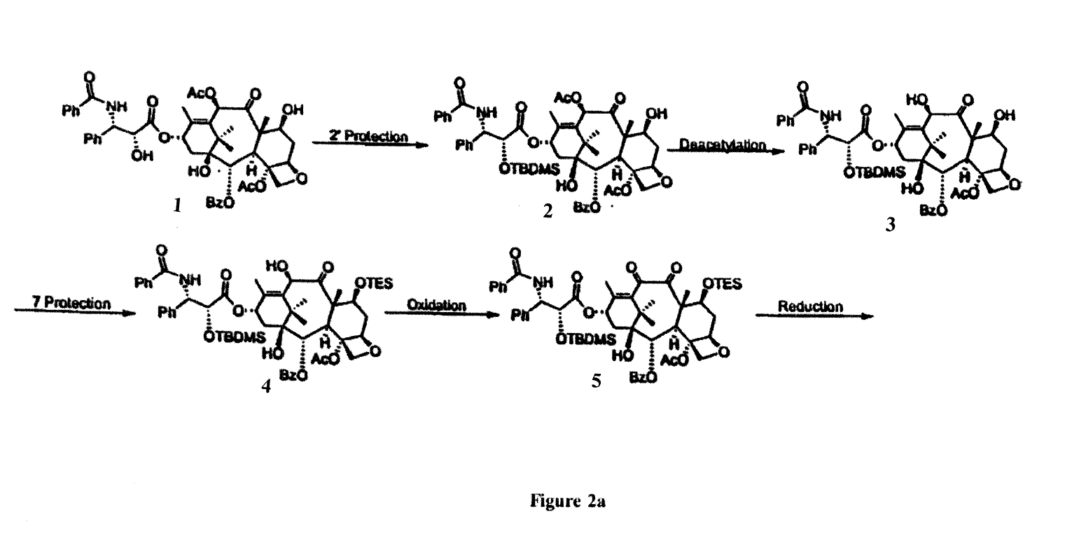

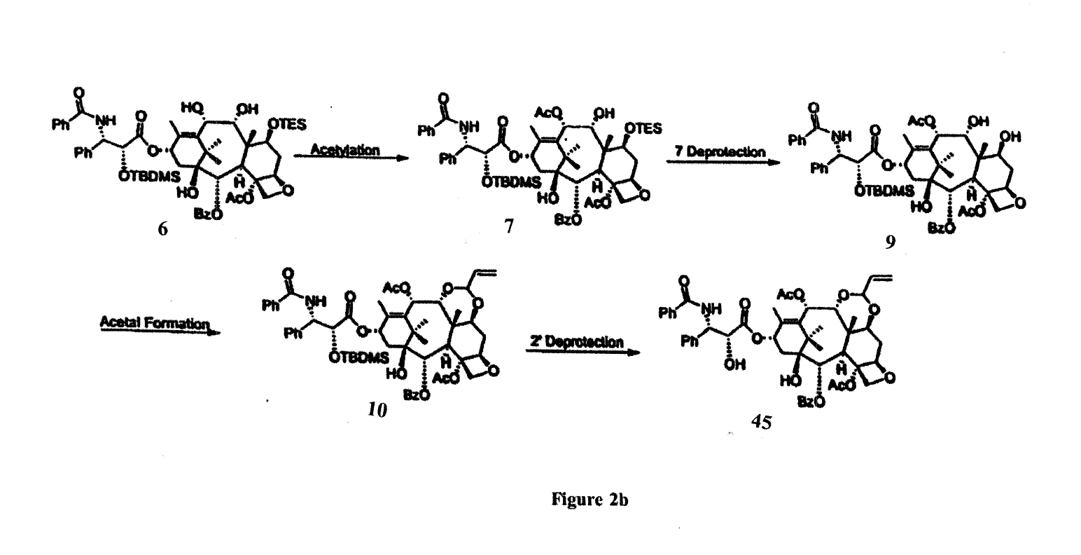

Such a process is exemplified in FIG. 2. For example, as shown, paclitaxel of Formula 1 is first protected at the 2′-hydroxyl with a hydroxyl protecting group such as tert-butyldimethylsilyl (TBDMS). To a 500 mL round bottom flask (RBF) equipped with a magnetic stir bar was charged 50.0 g (58.6 mmol) paclitaxel, Formula 1, 14.0 g (205 mmol, 3.5 eq.) imidazole, and 26.5 g (176 mmol, 3.0 eq.) TBDMS-Cl. The flask was placed under a nitrogen environment and 350 mL (7 mL/g paclitaxel) anhydrous N,N-dimethyl formamide (DMF) was charged to the flask. The reaction was stirred at room temperature for twenty hours, then was worked up by diluting the reaction solution in 600 mL isopropyl acetate (IPAc) and washing with water until the aqueous wash reached pH 7, then with brine. The organic partition was dried over magnesium sulfate, filtered and then was evaporated to a white foam solid to yield 66.9 g (93.0 area percent) of unpurified 2′-O-TBDMS paclitaxel product of Formula 2.

Next, the 10-acetyl group is removed using methods known in the art, such as by hydrazinolysis. To a 1 L REF equipped with a magnetic stir bar was charged 59.5 g 2′-O-TBDMS paclitaxel of Formula 2 and 600 mL (10 mL/g) IPM. The solution was stirred to dissolve the 2′-O-TBDMS paclitaxel, then 60 mL (1 mL/g) hydrazine hydrate was charged to the flask and the reaction stirred at room temperature for one hour. The reaction was worked up by diluting the reaction solution in 1.2 L IPAc and washing first with water, then ammonium chloride solution, then again with water until the aqueous wash was pH 7 and lastly with brine. The organic partition was dried over magnesium sulfate, filtered and evaporated to 55.8 g of solid. The solid was redissolved in 3:1 IPAc (1% water):heptne to a concentration 0.25 g/mL total dissolved solids (TDS) and purified on a YMC silica column; the column eluent was monitored for UV absorbance. The fractions were pooled based on HPLC analysis and evaporated to yield 39.3 g (98.6 area percent) of 2′-O-TBDMS-10-deacetylpaclitaxel solid of Formula 3.

The 7-hydroxyl is further protected with a protecting group such as triethylsilyl (TES). To a 500 mL RBF equipped with a magnetic stir bar was charged 39.3 g (42.5 mmol) 2′-O-TBDMS-10-deacetyl paclitaxel of Formula 3 and 15.6 g (127 mmol, 3 eq.) 4,4-dimethylaminopyridine (DMAP). The flask was placed under nitrogen and 390 mL (10 mL/g) anhydrous dichloromethane (DCM) was charged to the flask to dissolve the solids followed by 14 mL (84.9 mmol, 2 eq.) TES-Cl, The reaction was stirred at room temperature for three hours. The reaction was worked up by evaporating the reaction solution to approximately half its starting volume and diluting it in 300 mL EtOAc and washing with water and dilute HCl solutions until the pH of the aqueous wash was approximately 7, then washing with brine. The organic partition was dried over magnesium sulfate and evaporated to yield 42.0 g (97.7 area percent) of white solid of Formula 4.

Next, oxidation of the 10-hydroxyl yields a 9,10-diketo compound. To a 1 L RBF equipped with a magnetic stir bar was charged 41.0 g (39.4 mmol) 2-O-TBDMS-7-O-TES-10-deacetyl paclitaxel of Formula 4, 2.1 g (5.92 mmol, 0.15 eq.) of tetrapropylammonium perruthenate (TPAP), 13.9 g (118 mmol, 3 eq.) N-methylmorpholine-N-oxide (NMO). The flask was placed under nitrogen and 720 mL (˜20 mL/g) anhydrous DCM charged to the flask to dissolve the solids. The reaction was stirred at room temperature for 22 hours. The reaction was worked up by concentrating the reaction solution to half its volume and then drying the reaction contents onto 175 g silica gel (EM Sciences 40-63μ). The taxane containing silica was placed on 30 g of clean silica gel (EM Sciences 40-63μ) and the product eluted from the silica with 4 L methyl tert-butyl ether (MTBE). The MTBE was evaporated to yield 37.3 g (93.2 area percent) 2′-O-TBDMS-7-O-TES-9,10-diketo paclitaxel of Formula 5.

Selective reduction of the 9,10-diketo taxane yields the 9,10-α,α-hydroxy taxane. To a 2 L RBF equipped with a magnetic stir bar was charged 37.3 g (35.9 mmol) protected 9,10-diketo paclitaxel of Formula 5 and 900 mL (˜30 mL/g, taxane) of 3:1 EtOH/MeOH. The solution was stirred to dissolve the solids then the flask was placed in an ice/water bath and the solution was stirred for 30 minutes. Then 8.1 g (216 mmol, 6 eq.) of sodium borohydride (NaBH₄) was charged to the flask and the reaction stirred in the Ice/water bath for five hours. The reaction was worked up by diluting the reaction solution in 1 L IPAc and washing with 4×750 mL water, then with 200 mL brine. The organic partition was dried over magnesium sulfate. The aqueous washes were reextracted with 500 mL IPAc. The organic reextract solution was washed with 100 mL brine then dried over magnesium sulfate and combined with the first organic partition. The IPAc solution was concentrated until solids began precipitating out then heptane was added to the solution to crystallize the protected 9,10-α,α-OH, 9-desoxo, 10-deacetyl paclitaxel product of Formula 6. The crystallizing solution was placed in a freezer overnight. Three crystallizations were done on the material, the first yielded 4.1 g (95.3 area percent) protected 9,10-α,α-OH, 9-desoxo, 10-deacetyl paclitaxel product, the second yielded 18.3 g (90.9 area percent) product, and the third yielded 2.9 g (81.7 area percent) product. The original work on this reaction employed flash chromatography to purify the product. However, the crystallizations that were performed gave similar purity, by HPLC, to the chromatographed material from earlier work.

To a 25 mL RBF, equipped with a magnetic stir bar and under a nitrogen environment, was charged 300 mg (0.288 mmol) of 2′-O-TBDMS-7-O-TES-9,10-α,α-OH, 9-desoxo, 10-deacetyl paclitaxel of Formula 6, (0.720 mmol, 2.5 eq.) acid chloride (CH₃COCl), 140 μL (1.01 mmol, 3.5 eq.) triethyl amine (TEA), 13 mg (0.086 mmol, 0.3 eq.) 4-PP, and 10 mL anhydrous DCM. The reactions were stirred at room temperature for 15+ hours; reactions generally ran overnight and were monitored by TLC and/or HPLC in the morning for consumption of starting material. The reactions were worked up by diluting the reaction solution in 20 mL EtOAc and washing with water until the pH of the water washes was approximately 7. The organic solution was them washed with brine and dried over sodium sulfate before evaporating to dryness. The resulting product is the 2′-O-TBDMS-7-O-TES-9-α-OH,9-desoxo,10-epi paclitaxel of Formula 7 (where R₁═R₂=Ph; P₁=TBDMS; P₂=TES; R₇═CH₃ in generalized formula G of FIG. 1).

There are numerous alternative groups that may be used for the R₇COO group at the 10-α-position of generalized formula G. As would be appreciated by one skilled in the art, these acylation reactions may be performed for example by substituting the appropriate carboxylic acid R₇COOH, carboxylic acid halide R₇COX or carboxyl anhydride R₇COOCOR₇ (symmetrical or mixed anhydride) for example, in the above procedures.

When the reagent used is a carboxyl anhydride, an exemplary procedure is as follows. To a 25 mL RBF, equipped with a magnetic stir bar and under a nitrogen environment, was charged 300 mg (0.288 mmol) 2′-O-TBDMS-7-O-TES-9,10-α,α-OH, 9-desoxo, 10-deacetyl paclitaxel of Formula 6, (2.88 mmol, 10 eq.) acid anhydride (CH₃COOCOCH₃), 106 mg (0.864 mmol, 3 eq.) DMAP, and 5 mL anhydrous DCM. The reactions were stirred at room temperature for 15+ hours. The reactions were worked up by adding 5 mL saturated sodium bicarbonate solution to the reaction flask and stirring for 5 minutes. The solution was then transferred to a separatory funnel and organics were extracted with 20 mL EtOAc. The organic extract was then washed with saturated sodium bicarbonate and water until the pH of the water washes was approximately 7. The organic partition was then washed with brine and dried over sodium sulfate before evaporating to dryness.

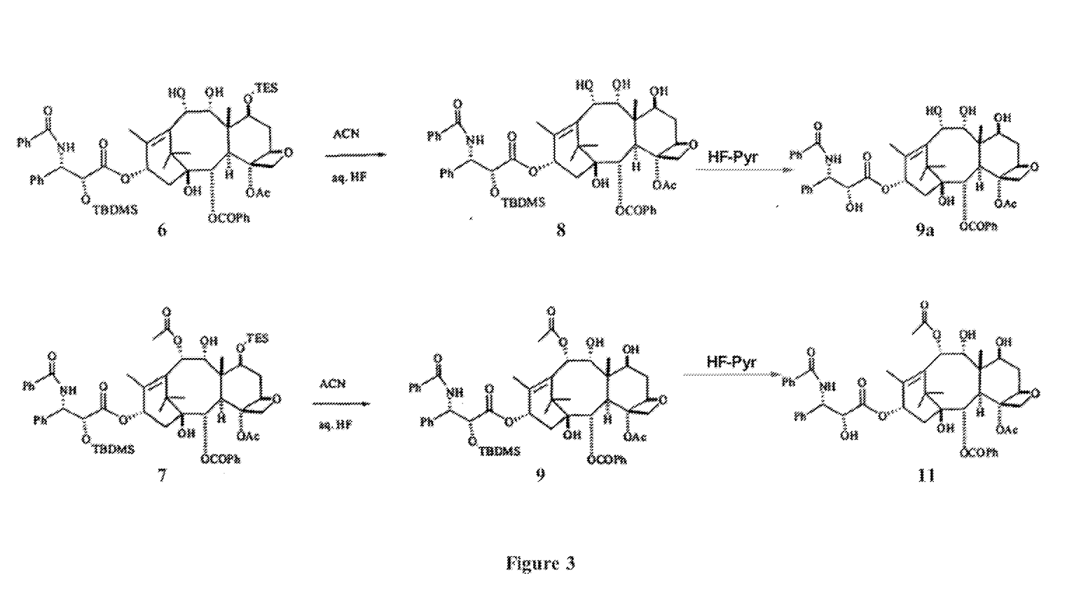

Taxanes of generalized formula G may be deprotected at the 2′- and 7-positions in either a two-step process or a single step. For example, as shown in FIG. 3, the 7-O-TES group may be removed from Formula 6 to give Formula 8 or from Formula 7 to give Formula 9, respectively, using acetonitrile (ACN) and aqueous HF. To a 500 mL teflon bottle equipped with a magnetic stir bar was charged 2.50 g (2.40 mmol) 2′-O-TBDMS-7-O-TES-9.10-α,α-OH, 9-desoxo, 10-deacetyl paclitaxel of Formula 6 and 100 mL ACN. The bottle was placed in and ice/water bath and the solution was stirred for 30 minutes. Next, 0.8 mL of 48% HF aqueous was added slowly to the reaction solution and the reaction stirred in the ice/water bath for 20 minutes. The reaction was monitored by TLC for disappearance of the starting material. The reaction was worked up by diluting the reaction solution by adding 200 mL EtOAc and quenching the acid by adding 25 mL saturated sodium bicarbonate solution to the bottle and stirring for 10 minutes. The solution was then transferred to a separatory funnel and the organic partition was washed with water until the pH of the water wash was approximately 7, then was washed with brine. The organic partition was dried over sodium sulfate and then was evaporated to a solid of Formula 8. This procedure was also followed if there was an acyl group on the 10-α-hydroxyl (i.e. Formula 7 to Formula 9 in FIG. 2).

Next, the 2′-O-protecting group may be removed from Formula 8 to give Formula 10 or from Formula 9 to give Formula 11, respectively, as shown in FIG. 3. To a 50 mL teflon bottle equipped with a magnetic stir bar was charged, 500 mg 2′-O-TBDMS-9,10-α,α-OH, 9-desoxo, 10-deacetyl paclitaxel of Formula 8 (or 2′-O-TBDMS-9-α-OH, 9-desoxo,10-epi paclitaxel of Formula 9) and 5 mL anhydrous THF. Next, 1 mL HF-pyridine solution was slowly charged to the reaction solution. The reaction was stirred at room temperature for 1 hour; reaction progress was monitored by TLC and/or HPLC for disappearance of starting material. The reaction was worked up by adding 10 mL EtOAc to the bottle to dilute the reaction solution and then saturated sodium bicarbonate was slowly added to the bottle to neutralize the HF. The solution was transferred to a separatory funnel and the organic partition was washed with 10 wt % sodium bicarbonate solution then water until the pH of the water wash was approximately 7. Then the organic partition was washed with brine and then dried over sodium sulfate before evaporating to a solid of Formula 9a (or Formula 11).

Further, as indicated above, the 2′- and 7-positions of either the taxanes of the generalized formula F or G may be deprotected in a one-step procedure using tetrabutylammoniumfluoride (TBAF). For example, Formula 6 may he deprotected directly to Formula 9a, and Formula 7 may he deprotected directly to Formula 11. A 10 mL RBF equipped with a magnetic stir bar was charged with 100 mg of 2′-O-TBDMS-7-O-TES-9-α-OH-OH, 9-desoxo, 10-deacetyl paclitaxel of Formula 6 (or 2′-O-TBDMS-7-O-TES-9-α-OH-10-epi paclitaxel of Formula 7) and 5 mL EtOAc or THF to dissolve the taxane. Next, 100 μl of 1M TBAF in THF was charged to the flask and the reaction was stirred at room temperature for 1 hour; the reaction was monitored by TLC and/or HPLC for disappearance of starting material. The reaction was worked up by washing the reaction solution with water and then brine. The organic partition was dried over sodium sulfite and evaporated to a solid of Formula 9a (or Formula 11). This method removes both the 2′-O-TBDMS protecting group and the 7-O-TES protecting group.

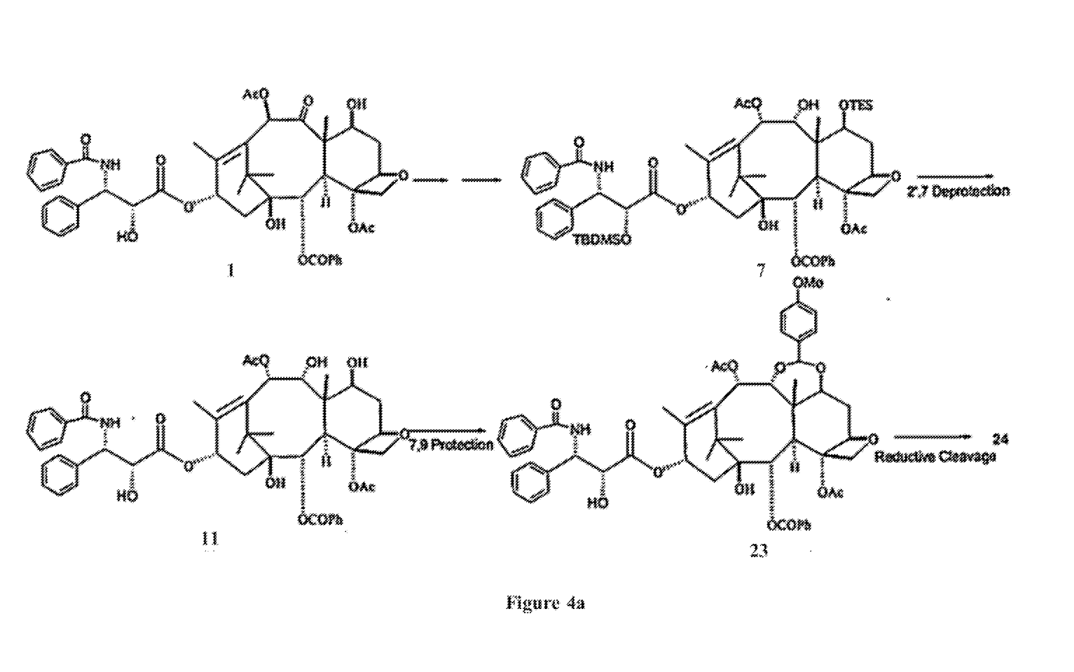

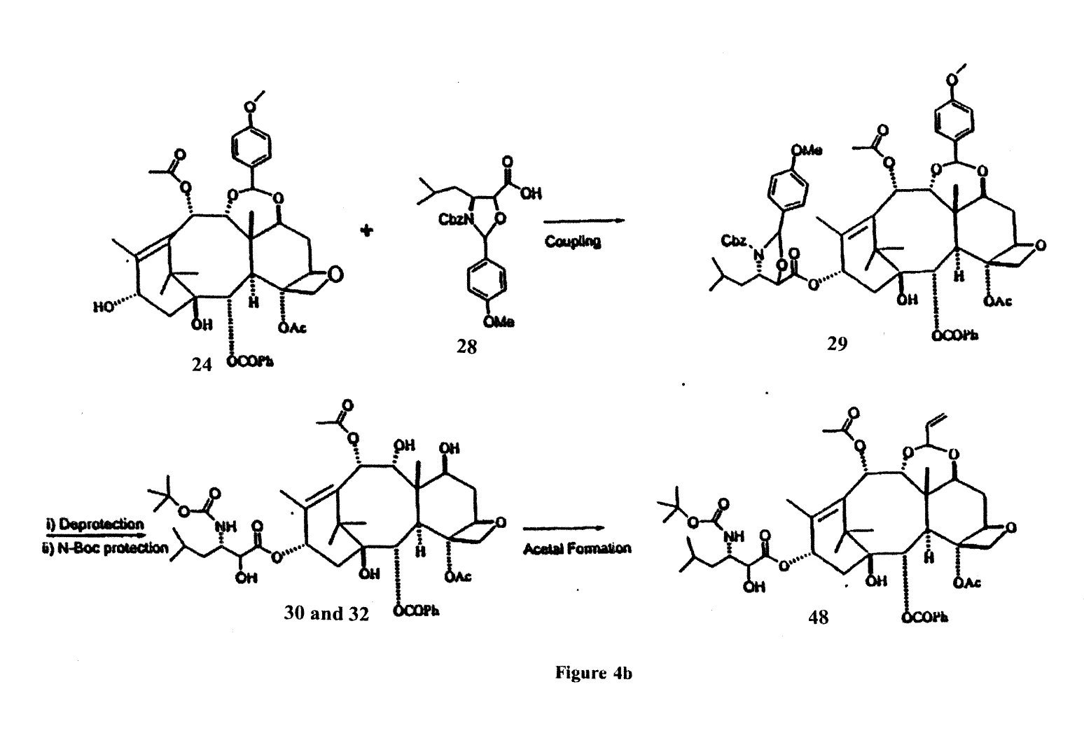

As shown for example in FIG. 4, the compound of Formula 11 may be protected as a 7,9-acetal, such as a cyclic acetal such as with anisaldehyde dimethyl acetal to form a compound of Formula 23 (where R₁═R₂=Ph; R₇═CH₃; R₈═H; R₉-PhOMe in generalized formula M of FIG. 1). To a 50 mL RBF was charged 1.15 g (1.35 mmol) 9-α-OH-9-desoxo-10-epi paclitaxel of Formula 11 and 25 mL anhydrous DCA, under nitrogen, 343 μL (2.02 mmol, 1.5 eq.) anisaldehyde dimethyl acetal was charged to the flask, followed by 51 mg (0.269 mmol, 0.2 eq.) p-toluenesulfonic acid (PTSA). The reaction was stirred at room temperature for 45 minutes then was worked up by extracting the product with EtOAc and washing with saturated sodium bicarbonate solution followed by water. The organic partition was evaporated to yield approximately 1.5 g of crude product. The crude product was purified by flash chromatography to yield 0.72 g of pure product of Formula 23.

Next, the side chain is cleaved to form the compound of Formula 24, as exemplified in FIG. 4. To a 25 mL RBF was charged 720 mg (0.740 mmol) 9-desoxo-7,9-anisaldehyde acetal-10-epi paclitaxel of Formula 23 and 15 ml., anhydrous THF, under nitrogen. The flask was placed in an ice/water/ammonium chloride, −13° C. bath. Solid lithium borohydride (29.0 mg, 1.33 mmol, 1.8 eq.) was charged to the reaction flask and the reaction stirred at −13° C. for two hours before raising the temperature to 0° C. The reaction was worked up after five hours fifteen minutes by diluting with EtOAc and washing with water and ammonium chloride solution. The organic partition was evaporated to yield 650 mg of crude compound but HPLC indicated that there was only approximately 20% product and mostly unreacted starting material; therefore, the reaction was restarted by repeating the above procedure and running the reaction for an additional six hours. The organic partition was evaporated to yield approximately 660 mg of crude product. The compound was purified on a spherical silica column to yield the compound of Formula 24.

FIG. 4 provides the coupling reaction of Formula 24 with Formula 28 to provide the compound of Formula 29. To a 5 mL RBF was charged 180 mg (0.255 mmol) 7,9-anisaldehyde acetal, 9-desoxo 10-epi Baccatin III (Formula 24) and 105 mg (0.510 mmol, 2.0 eq.) DCC. Toluene (2 mL) was then added to dissolve the solids. Next, Formula 28 (158 mg, 0.383 mmol, 1.5 eq.) was dissolved in 1.0 mL DCM and the solution was charged to the reaction flask followed by 6 mg (0.038 mmol, 0.15 eq.) 4-PP. The reaction was stirred at room temperature for 23 hours and then was quenched by adding 11.5 μL acetic acid and 4 μL water and stirring for one hour. MTBE was added to the reaction flask to precipitate DCU and the reaction solution was filtered to remove the precipitate. The filtrate was slurried with activated carbon then passed across a silica plug to remove the 4-Pp salts, The eluent was evaporated to a solid to yield 271 mg of crude coupled product of Formula 29.

As further exemplified in FIG. 4, the 7,9-acetal and N,O-acetal protecting groups may then be removed and an N-acyl group added to form the compounds of Formula 30 and 32 (where R₁=t-butoxyl; R₂═CH₂CH(CH₃)₂; R₇═CH₃ in generalized formula L of FIG. 1), which may be separated from each other by liquid chromatography or kept together for the next step. While the same anisaldehyde group is used at both the 7,9-acetal and N,O-acetal in the exemplary compound of Formula 29, such that both groups may be removed in a single step, it should be appreciated that other acetal protecting groups are contemplated such that multiple deprotection steps may be required. To a 10 mL RBF was charged, 270 mg (0.245 mmol) of 7,9-anisaldehyde acetal-10-epi-3′-isobutyl-3′,2′-N,O-anisaldehyde acetal coupled ester of Formula 29, 220 mg (0.8 g/g coupled ester) Degussa type palladium on carbon, and 4.1 mL THF. In a separate vial, 99 μL conc., HCl was diluted in 198 μL water and 1.0 mL THF. This solution was added to the reaction flask and the flask was sealed and placed under hydrogen. The hydrogenation reaction was stirred for 31 hours then was quenched by removing the hydrogen and filtering the catalyst from the reaction solution then adding 84.5 μL (0.368 mmol, 1.5 eq.) t-butoxy carbonyl (t-BOC) anhydride followed by 684 μL TEA. The reaction stirred an additional 21 hours and then was worked up, diluting the filtrate with EtOAc and washing with water. The organic partition was evaporated to approximately 370 mg of oil. The oil was purified first by flash chromatography, then preparative TLC (PTLC) then by a semi-prep reverse phase column to yield 3.9 mg of pure product of Formula 30 and 32.

An alternate 7,9-acetal may be formed if desired to provide the compound of Formula 31 or 33 (where R₁ is t-butoxyl; R₂ is CH₂CH(CH₃)₂; R₇ is CH₃; R₈ is H; R₉ is CH═CH₂ in generalized formula M of FIG. 1). In a HPLC vial insert, 3.4 mg (4.13 μmol) of the taxanes of Formula 30 and 32 was charged followed by 70 μL DCM. Next, 12.8 μL of a 1 to 20 diluted acrolein dimethyl acetal in DCM (0.64 μL acetal, 5.37 μmol, 1.3 eq.) was charged to the insert followed by 8.4 μL (0.413 μmol, 0.1 eq.) of a 0.05M PTSA solution in DCM. The reaction was lightly agitated then sat at room temperature. The reaction took more additions of the acetal solution to drive it to completion then was worked up after a couple of days by filtering the solution through approximately 80 mg of basic activated alumina. The alumina was washed with DCM then EtOAc and the fractions evaporated to dryness. The crude compound was purified on a normal phase analytical column to yield 605 μg of compound (the product was an isomeric mixture) taxanes of Formulae 31 and 33.

As generally described and specifically exemplified above, these processes may be performed with the isolation of one or more of the intermediate compounds, or the process may be performed without the isolation and purification at each and every single processing steps.

### Separation of Diastereoisomers of Formula 48 by Normal Phase Chromatography

A solution of the mixture of isomers, 31 and 33, originally identified as Formula 48 in ACN (570 mg) was concentrated to light yellow oil, dried in the vacuum oven for 15 min and re-dissolved in 35:65 MTBE/n-heptane. The solution was loaded onto a flash chromatography column packed with spherical silica (YMC-1701, 56 g), which had been conditioned with 35:65 MTBE/n-heptane. The solution flask was rinsed (2×) with ˜2 mL of MTBE onto the column. The column was eluted with 35:65 MTBE/n-heptane and fractions (25 mL) were collected. Fractions containing the pure product (fr. 23-25) as indicated by visual spotting (to identify the elution of UV active material) and by TLC analysis (50:50 MTBE/n-heptane) were collected, pooled and concentrated to give 305 mg of S-31 as a white solid.

The compound S-31 was characterized by NMR, including ¹H, ¹³C, HBMC, HSQC, NOESY, COSY and gHSQMBC. The compound of Formula S-31 was also analyzed by β-tubulin binding modeling studies. Similarly compound R-31 was also characterized by NMR, including ¹H, ¹³C, HMBC, HSQC, NOESY, COSY and gHSQMBC.

### In Vitro ED₅₀ MT Polymerization Study

In this tubulin binding assay, microtubule protein (MTP) is used as a substrate. The assay contains bovine tubulin plus microtubule associated proteins (MAP). MTP is polymerized into microtubules in the presence of DAN (4′,6′-diamidino-2-phenylindole), a fluorescent compound. DAN binds to rubulin; when microtubules are formed and there is an enhancement of fluorescence. The microtubule formation is measured as a function of time, using a fluorescence plate reader. The ED₅₀ values obtained with this method are in good agreement with older sedimentation techniques. The more current assay, using DAPI, is faster and uses less protein. The method used is based on the procedure published by Donna M. Barron, et al, “Fluorescence-based high-throughput assay for antimicrotuble drugs” Analytical Biochemistry, 315; 49-56, 2003, which is incorporated by reference in its entirety. The excitation wavelength, in that assay, was set at 370 nm and the emission wavelength was set at 450 nm for the DAPI experiments.

A Bio-Tek FL 600 microplate Fluorescence Reader was used to measure the relative level of fluorescence in the DAPI assay.

Assays were conducted in 96-well plates. Each well contained a total volume of 0.1 mL consisting of PEM buffer (0.1 M Pipes, 1 mM EGTA, 1 mM MgCl₂, pH 6.9), 0.2 mg bovine microtubule protein, and 10 μg of DAPI. Compounds having paclitaxel-like activity of varying concentrations dissolved in DMSO were added last. The final DMSO concentration was 4%. The plates were incubated at 37° C. for 30 minutes and read in a fluorescence plate reader using an excitation wavelength of 360 nm and an emission wavelength of 460 nm. Fluorescence values were corrected for the sample without compound. Results were expressed as a percent of maximum assembly, with maximum assembly taken to be that obtained at 25 μM paclitaxel.

Experiments were done twice in triplicate. Results were subsequently combined and fit to a non-linear regression program.

The results from these studies summarized in Table 4 indicate that S-31 has an ED₅₀ potency that is equal to or greater than that determined for other tubulin binding agents such as paclitaxel, docetaxel, and Epothilone B.

TABLE 4
Summary of Tubulin Polymerization Assays, Comparison
of TPI 287 to paclitaxel, docetaxel, and epothilone B.

| Compound | ED50, μM | ED50 Compound/ED50 Paclitaxel |
|---|---|---|
| S-31 | 1.58 ± 0.46 | 0.53 |
| Paclitaxel | 2.97 ± 0.50 | 1.00 |
| Docetaxel | 3.18 ± 0.45 | 1.07 |
| Epothilone B | 3.31 ± 0.51 | 1.11 |

### Alternative Method for Synthesizing 7,9-Acetal Linked Analogs

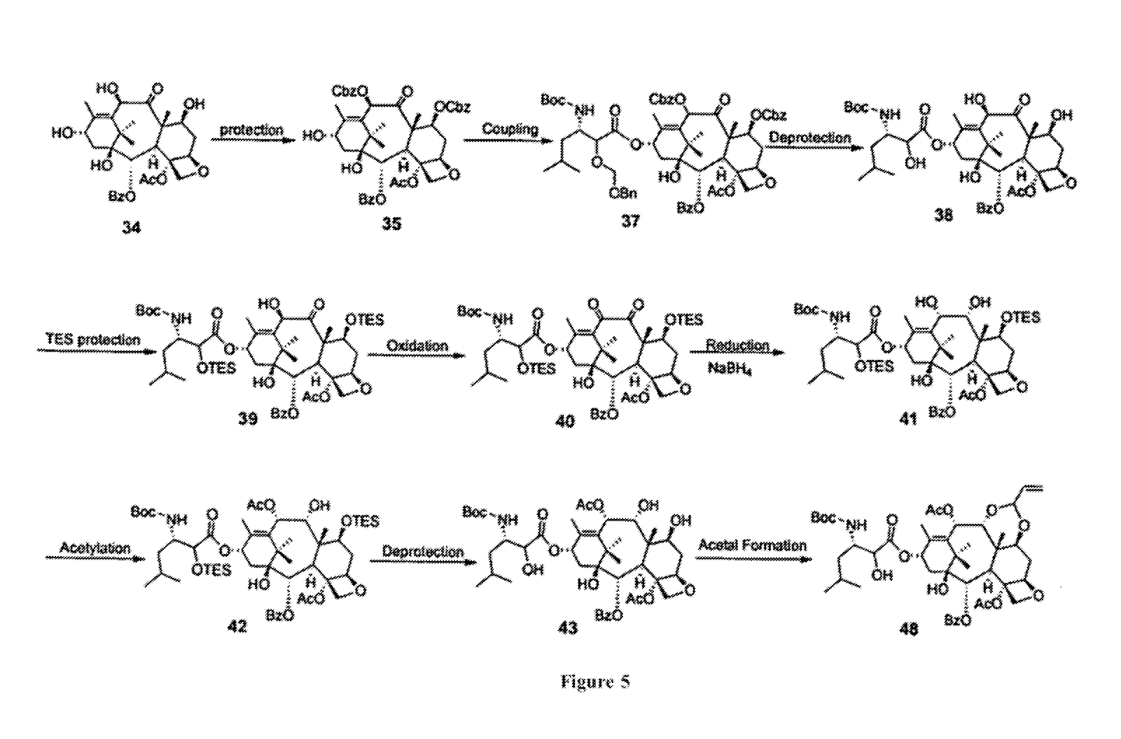

7,9 Acetal linked analogs of 9,10-α,α OH taxanes can also be formed directly from 10-deacetylbaccatin III (10-DAB III), which has Formula 34 as shown in FIG. 5,

Using 10-DAB has an advantage since it is much more naturally abundant and thus less expensive than the starting compound A shown and discussed in FIG. 1.

In this alternative process, 10-DAB III, Formula 34, is first protected at both the C-7 and C-10 positions to form C7,C10 di-CBZ 10-deacetylbaccatin III, Formula 35. 10-deacetylbaccatin III of Formula 34 (50 g, 91.8 mmol) was dissolved in THF (2 L, 40 ml/g) by warming to 40° C. in a warm-water bath. The solution was cooled to −41° C. in a Neslab chiller and benzylchloroformate (46 mL. 3.2 eq, 294 mmol) was added to the stirred chilled solution followed by further cooling to −44° C. To this solution 23 M hexyl lithium solution (130 mL, 3.3 eq, 303 mmol) was added gradually over 45 min while maintaining the temperature of the reaction mixture at ≦−39° C. Stirring continued in the Neslab for 45 minutes at which time HPLC indicated the reaction had gone to completion. At 2 hr total reaction time, the reaction was quenched by the addition of 1N HCl (400 mL) and IPAc (1 L) and removal from the Neslab chiller. The reaction was allowed to stir while warming to 10° C. The layers were separated and the IPAc layer was washed sequentially with H₂O (500 mL), saturated NaHCO₃ (200 mL) and H₂O (4×500 mL) and then filtered through a silica gel pad. The filtrate was concentrated until solids started to form, IPAc (850 mL) was added and the mixture was heated to 60° C. to dissolve some of the solids. To the warm solution, heptanes (800 mL) were added and the solution was cooled in the refrigerator and filtered. The solids collected by the filtration were washed with heptanes and dried under vacuum at 45° C. to give Formula 35.

Next, Formula 35 was coupled with a side chain to form Formula 37. Here, the side chain of Formula 36, (38 g, 99.6 mmol) was dissolved in toluene to a known concentration (0.0952 g/mL). This solution was added to Formula 35 (54.0 g, 66.4 mmol). The solution was heated in a warm-water bath and DMAP (8.13 g, 66.4 mmol) and DCC (25.3 g, 320 mmol) in toluene (540 mL) were added to the warm reaction mixture. While maintaining the temperature it about 51° C., the reaction was continually stirred and sampled periodically for HPLC. After 3 hours, additional DCC (13.0 g) in toluene (140 mL) was added. The following morning (25.25 hr), MTBE (450 mL) was added and the reaction mixture was filtered through a pad of silica gel, washed with MTBE followed by EtOAc, and concentrated to give Formula 37 as 61.8 g of an oil,

Formula 37 was then deprotected at both the C7 and C10 position to give Formula 38. A solution of THF (300 mL) and HCl (22 mL) was added to a solution of Formula 37 (61.8, 52.5 mmol) in THF (15 mL/g, 920 mL). The resulting solution was flushed with nitrogen. A catalyst (10% Pd/C with 50% water, 99.1 g) was added and the flask was flushed with nitrogen three times and then with hydrogen three times. The reaction mixture was stirred vigorously under a hydrogen balloon for 21 hours. At this time the reaction was sampled and HPLC indicated that 38% by area of starting material still remained. Water (10 was added and stirring continued. Twenty hours later, HPLC indicated the same amount of starting material still remaining. The reaction mixture was filtered through celite and washed with THF. It was then concentrated to remove excess THF; fresh catalyst (101 g) was added and the reaction mixture was placed back under hydrogen as before. After another 24 hours, an intermediate compound was still present and still more catalyst (20 g) was added. After another hour, HPLC indicated that the reaction was complete. The reaction mixture was filtered through celite and washed through with IPAc. The combined filtrate was washed with NH₄Cl solution (500 mL), water (500 mL), 5% NaHCO₃ (500 mL), H₂O (300 mL), and brine (300 mL). The organic layer was dried, filtered, and concentrated to give a foam of Formula 38 (42.5 g).

Formula 38 was then converted to Formula 39. Formula 38 (41.4 g, 52.5 mmol) was dissolved in DCM (500 mL) at room temperature. In the case that the impurity was water, NaSO₄ was added to the solution, and the solution was filtered through filter paper into to a 2 L flask. The solids were collected and washed with DCM (250 and the washings transferred into the flask. The flask was covered with a septum and N₂ balloon. TEA (35 mL) followed by DMAP (1.28 g) and TES-Cl (˜30 mL, 3.5 eq) were added to the solution and stirred. Additional TES-Cl (15 mL) and TEA (20 mL) were added, and after 6 hours HPLC indicated the reaction had gone to completion.

The reaction was then quenched by the addition of EtOH (25 mL). The layers were separated and the organic layer was washed with saturated NH₄Cl (˜500 mL). The organic layer was dried over Na₂SO₄ and concentrated. A flash column was packed with silica gel and wet with 8:2 heptane/IPAc (1.5 L). The solids were dissolved in 8:2 heptane/IPAc (250 mL) and filtered to remove solids that would not dissolve. This solution was concentrated to ˜100 mL and applied to the column. The column was eluted with 8:2 heptane/IPAc and fractions collected. Fractions with product were pooled and concentrated to give foam of Formula 39 (24.5 g).

Formula 39 was then oxidized to form Formula 40. Here, solid Na₂SO₄ was added to a solution of Formula 39 (24.5 g, 24.0 mmol) and 4-methyl morpholine N-oxide (10.1 g, 84 mmol) in DCM (340 mL) to assure that the reaction was dry. The mixture was stirred for 1 hour and then filtered through 24 cm fluted filter paper into a 2L 3-N round bottom flask. The Na₂SO₄ solids were washed with DCM (100 mL) and the washings transferred into the flask. Molecular sieves (6.1 g, 0.15 g/g) were added to the solution and stirring was begun. TPAP (1.38 g) was added and the reaction was allowed to stir under a N₂ blanket. Samples were taken periodically for HPLC, Additional TPAP (0.62 g) was added after 2 hours and again (0.8 g) after 15 hours. The reaction mixture was applied to a pad of silica gel (86 g), wet with 8:2 heptane/IPAc and eluted with IPAc. The fractions were collected, pooled and concentrated to an oil. 4-Methyl morpholine N-oxide (5.0 g) and DCM (100 mL) were added and stirred. Na₂SO₄ (13 g) was added to the mixture and it was filtered through filter paper. The Na₂SO₄ solids remaining in the filter was washed with DCM (45 mL). Molecular sieves (5 g) and TPAP (1.03 g) were added to the solution and after 45 minutes, more TPAP (1.05 g) was added. A pad of silica gel was prepared and wet with 80:20 Heptane/IPAc. The reaction mixture was applied to the pad and eluted with IPAc. Fractions were collected and those fractions containing product were pooled and concentrated to give an oil product of Formula 40 (21.8 g).

Next, Formula 40 was reduced to form Formula 41. NaBH₄ (365 mg, 6 eq) was added to a stirred solution of Formula 40 (1.6 g) in EtOH (19 mL) and MeOH (6.5 mL) cooled in an ice-water bath. After 1 hour, the reaction mixture was removed from the ice-water bath and at 2 hours, the reaction was sampled for HPLC, which indicated the reaction had gone to completion. The reaction mixture was cooled in an ice-water bath and a solution of NH₄OAc in MeOH (15 mL) was added followed by the addition of IPAc (50 mL) and H₂O (20 mL). It was mixed and separated. The organic layer was washed with water (20 mL) and brine (10 mL), a second time with water (15 mL) and brine (10 mL), and then twice with water (2×15 mL). It was dried over Na₂SO₄ and placed in the freezer overnight. The following morning a sample was taken for HPLC and the reaction was dried and the organic layer was concentrated on the rotovap. It was placed in the vacuum oven to give a foam product of Formula 41 (1.45 g).

Formula 41 was then acylated to form Formula 42, TEA (5.8 mL, 41.5 mmol), Ac₂O (2.62 mL, 27.7 mmol) and DMAP (724 mg, 5.5 mmol) were added to a solution of Formula 41 (14.1 g, 13.8 mmol)) in DCM (50 mL). The reaction was stirred and sampled for HPLC periodically. After 18.5 hours, additional TEA (1.5 mL) and Ac₂O (1 mL) were added. At 19 hours, HPLC indicated the reaction had gone to completion. The reaction mixture was diluted with IPAc (300 mL) and poured into 5% NaHCO₃ (100 ml). It was then stirred, separated, and the organic layer was washed with water (100 mL), saturated NH₄Cl (2×100 mL), water (3×50 mL) and brine (50 mL) and then filtered through Na₂SO₄. The mixture was concentrated to give a foam product of Formula 42 (14.6 g).

Next, Formula 42 was converted to a compound of Formula 43. A quantity of Formula 42 (3.0 g, 2.83 mmol) was weighed into a 100 mL flask. Next, DCM (24 mL) followed by MeOH (6 mL) were added to the flask at room temperature. Stirring of the mixture began under N₂ and camphorsulfonic acid (CSA) (0.0394 g, 0.17 mmol) was added. After 4 hours LCMS indicated the product had formed. 5% NaHCO₃ (15 mL) was added to the reaction mixture; it was shaken vigorously and then transferred to a separator funnel. The reaction flask was rinsed into the separatory funnel with 5% NaHCO₃ (25 mL) and, thereafter, the reaction mixture was shaken and the layers were separated. The organic layer was washed with brine, dried over Na₂SO₄, and concentrated. MTBE (3×25 mL) was added and the reaction mixture was concentrated to dryness after each addition to finally give 3.71 g foam. The foam was dissolved in MTBE (10 mL) and stirred. Heptane (50 mL) was slowly added to the reaction solution and solids began to form immediately. The solids were vacuum filtered and rinsed with heptane (720 mL). The solids were collected and dried in a vacuum oven at 40° C. to give Formula 43 (2.18 g).

Formula 43 was then converted to Formula 48. A solution of Formula 43 (2.1 g, 2.52 mmol) in DCM (10.5 mL) was stirred at room temperature. Next, 3,3-dimethoxy-1-propene (2.03 g, 17.7 mmol) followed by CSA (0.035 g, 0.15 mmol) were added to the solution. After the solution was stirred for 3.5 hours, LCMS indicated the reaction had gone to completion. The reaction was diluted with DCM (25 mL) and added to a separatory funnel with 55 mL 5% NaHCO₃ solution. The layers were separated and the aqueous layer was washed with DCM (25 mL). The two organic layers were combined, washed with brine, dried over Na₂SO₄ and concentrated. A flash chromatography column was packed with silica gel (230-400 mesh) and wet with 50:50 MTBE/heptane (1000 mL). The reaction mixture was dissolved in MTBE (10 mL), loaded on the column and doted with 50:50 MTBE/heptane. The fractions were collected, pooled, concentrated and dried in a vacuum oven at 50° C. to give product of Formula 48.

Standard procedures and chemical transformation and related methods are well known to one skilled in the art, and such methods and procedures have been described, for example, in standard references such as Fiesers' Reagents for Organic Synthesis, John Wiley and Sons, New York, N.Y., 2002; Organic Reactions, vols. 1-83, John Wiley and Sons, New York, N.Y., 2006; March J. and Smith M.: Advanced Organic Chemistry, 6^{th} ed., John Wiley and Sons, New York, N.Y.; and Larock R. C.: Comprehensive Organic Transformations, Wiley-VCH Publishers, New York, 1999. All texts and references cited herein are incorporated by reference in their entirety.

### MTS Proliferation Assay (Promega)

Day 1: Cells were plated in appropriate growth medium at 5×10³ per well in 100 ul in 96 well tissue culture plates, Falcon, one for each drug to be tested. Col 1 was blank; it contained no cells, just medium. The plates were incubated overnight at 37° C., 5% CO₂ to allow attachment.

Day 2: Added 120 ul growth medium in wells of 96-well “dilution plates” (one for each drug) and let sit in 37° C. incubator for about 1 hr.

Thawed DMSO drug stocks (usually at 10 nM). Each drug was diluted 6 ul into a tube with 3 ml growth medium, to 20 uM.

Aspirated medium from col 12 of a dilution plate; added 200-300 ul of 20 uM drug to wells of col 12. Made serial dilution down this 96-well plate: for a 1:5 dilution pattern, moved 60 ul from col 12 to col 11, mixed 4-5 times (using 8 place multi-pipettor), moved 60 ul to col 10, etc. stopping at col 3.

Moved 100 ul of medium+drug from dilution plate to a cell plate, i.e. col 1 from drug plate (blank=no cells) to col 1 of cell plate, etc. up to col 12. Col 2 contained cells with no drug. Col 3 had the lowest concentration of drug (0.005 nM) and col 12 had the highest drug concentration (10 uM).

Day 4 or 5: Terminated the assay 48 to 72 hrs after drug addition. Thawed MTS reagent; made up enough medium+MTS to cover all plates at 115 ul per well (100 ul medium+15 ul MTS). Aspirated medium drugs from cell plate; replaced with medium+MTS mix and incubated 1-6 hrs (37° C., CO₂), depending on cell type. When the color turned dark in control wells (col 2), and was still light in col 12, the absorbance at 490 nm was read on a plate reader; the results were used to calculate IC₅₀.

Accordingly, the present application has been described with some degree of particularity directed to the exemplary embodiments of the present application. It should be appreciated, though, that the present application is defined by the following claims construed in light of the prior art so that modifications or changes may be made to the exemplary embodiments of the present application without departing from the inventive concepts contained herein.

We claim:

1. A compound as a single diastereoisomer of the formula:

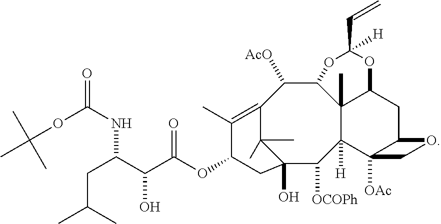

2. The compound of claim 1, wherein the compound is greater than 95% pure.

3. The compound of claim 1, wherein the compound is greater than 99% pure.

4. A pharmaceutical composition comprising:
a) a therapeutically effective amount of a compound of claim 1, in the form of a single diastereoisomer; and
b) a pharmaceutically acceptable excipient.

5. A method for the treatment of cancer in a patient comprising administering to the patient a therapeutically effective amount of a compound of the formula: 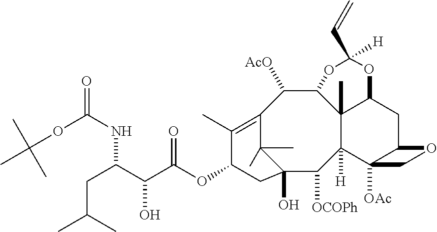
to a patient in need of such treatment.

6. The method of claim 5, wherein the cancer is selected from the group consisting of leukemia, neuroblastoma, glioblastoma, cervical, colorectal, pancreatic, renal and melanoma.

7. The method of claim 5, wherein the cancer is selected from the group consisting of lung, breast, prostate, ovarian and head and neck.

8. The method of claim 6, wherein the cancer is colorectal cancer.

9. The method of claim 6, wherein the cancer is pancreatic cancer.

10. The method of claim 6, wherein the cancer is neuroblastoma or a glioblastoma.
